(19) United States
(12) Patent Application Publication
(10) Pub. No.: US 2024/0392424 A1
(43) Pub. Date: Nov. 28, 2024

(54) DENTAL TITANIUM MATERIAL HAVING MODIFIED SURFACE, AND PREPARATION METHOD THEREFOR

(71) Applicant: Ajou University Industry-Academic Cooperation Foundation, Gyeonggi-do (KR)

(72) Inventors: Hee-Kyung KIM, Seoul (KR); Chul Ho KIM, Seoul (KR); Sung Un KANG, Gyeonggi-do (KR)

(73) Assignee: Ajou University Industry-Academic Cooperation Foundation, Gyeonggi-do (KR)

(21) Appl. No.: 18/699,240

(22) PCT Filed: Oct. 11, 2022
(86) PCT No.: PCT/KR2022/015305

§ 371 Date: Apr. 5, 2024

(30) Foreign Application Priority Data
Oct. 8, 2021 (KR) 10-2021-0134400

(51) Int. Cl. C22F 1/18; A61C 8/00; A61C 13/00

(52) U.S. Cl. CPC C22F 1/183; A61C 8/0012; A61C 13/0006; A61C 2008/0046

(57) ABSTRACT

The present invention relates to a dental titanium material having a surface modified through plasma treatment; an implant; and a preparation method therefor. The titanium material and the implant prepared according to the present invention have improved cell adhesion and antimicrobial effect so that biocompatibility is improved and the inflammatory response of a user and the like can be minimized.

### TECHNICAL FIELD

The present invention provides a titanium material having a surface modified by a method for improving the biocompatibility and antibacterial effect of titanium, and more particularly, provides a method of treating an implant made of titanium with plasma to improve the cell adhesion ability of the implant and to improve the antibacterial effect against harmful bacteria such as P.gingivalis.

Meanwhile, this application was supported by the following national research and development projects.
[National research and development projects that supported this invention] 1.
[Task identification number] 1465034377
[Task number] HR21C1003 (HR21C1003010021)
[Implementing Ministry] Ministry of Health and Welfare
[Research management agency] Ministry of Health and Welfare (Korea Health Industry Development Institute)
[Project name] Research-oriented hospital development (R&D)
[Task name] Establishment of Quattro win-win platform based on hyper-personalized H·I future technology of the 5th industrial revolution (Establishment of an early technology commercialization platform for customized healing innovation H·I (Human Interface))
[Host organization] Ajou University Medical Center
[Research period] Jul. 1, 2021˜Dec. 31, 2029
[Task identification number] 1711144175
[Task number] 2018R1A2B3009008
[Implementing Ministry] Ministry of Science and ICT
[Research management agency] National Research Foundation of Korea
[Project name] Individual basic research (Ministry of Science and ICT) (R&D)
[Task name] Development of a precise control treatment strategy to overcome treatment-resistant cancer through the analysis of tumor heterogeneity and tumor microenvironment based on single-cell transcriptome in refractory head and neck cancer
[Host organization] Ajou University
[Research period] Mar. 1, 2018˜Feb. 28, 2023 3.
[Task identification number] 1711144980
[Task number] 2019R1F1A1062112
[Implementing Ministry] Ministry of Science and ICT
[Research management agency] National Research Foundation of Korea
[Project name] Basic research
[Task name] Development of surface treatment technology to improve the function of high-transparency cubic-phase zirconia as a new dental material
[Host organization] Ajou University
[Research period] Jun. 1, 2019˜Aug. 31, 2022 4.
[Task identification number] 1711172489
[Task number] 2022RIF1A1067929
[Implementing Ministry] Ministry of Science and ICT
[Research management agency] National Research Foundation of Korea
[Project name] Basic research
[Task name] Development of critical technology to improve the function of dental high-transparency zirconia through plasma ion nitriding method
[Host organization] Ajou University
[Research period] Jun. 1, 2022˜Feb. 28, 2025

### BACKGROUND ART

Dental implants (hereinafter simply referred to as ‘implants’) are artificial teeth that can permanently replace missing teeth, so they must be able to functionally play the role of actual teeth. In addition, they should be manufactured so that they can be used for a long time by properly distributing the load applied to the teeth during mastication, and are also required to be delicately made to have a shape and color that are not much different from actual teeth in terms of beauty.

The implants are transplanted and fixed into biological tissues in the oral cavity, that is, alveolar bones. As time passes after being implanted in the living body, the metal ions of the metal implant are eluted by tissue fluid or body fluid in the body or due to contact and friction with the biological tissues, thereby corroding the implants. In addition, the metal ions eluted from the metal implant may damage macrophages in the living body or invade cells in the living body to generate inflammatory cells or giant cells, so the implants must have excellent biocompatibility.

Although the materials of these implants have been attempted to be developed from various metals and alloys, titanium metal or its alloys, which have the advantages of high mechanical strength and bio-inactivity, are mainly used. However, the titanium has the disadvantage of being weak in initial osseointegration and masticatory power, making it prone to early dislodgement when implanted in the body, and to improve this, research is being conducted on coating the surface of titanium implants by applying various surface treatment methods.

Meanwhile, the process of placing the implant in the body is performed through a surgical operation, and problems due to bacterial infection and inflammation frequently occur during the surgical operation. Therefore, there is a need for research on methods capable of improving cell adhesion ability while increasing the antibacterial effect of implants.

### DISCLOSURE

### Technical Problem

The present invention is intended to provide a dental titanium material or a dental titanium implant that is surface modified to have improved chemical activity, cell adhesion ability, and antibacterial effect.

The present invention is to provide a titanium implant with improved stability, antibacterial property, cell proliferation, cell mobility, and cell adhesion by discovering plasma that is effective in improving the cell adhesion ability and antibacterial effect of titanium materials and dental titanium implants, and treating the titanium with this plasma.

The present invention also aims to provide a plasma treatment process for manufacturing a surface-modified titanium material and a dental titanium implant.

However, the technical problems to be achieved by the present invention are not limited to the above-mentioned problems, and other problems not mentioned will be clearly understood by those skilled in the art from the following description.

### Technical Solution

Hereinafter, the present invention will be described in detail. The advantages and features of the present invention will become clear with reference to the embodiments described below for achieving them. However, the present invention is not limited to the embodiments described below and may be implemented in various different forms. These embodiments are provided only to make the disclosure of the present invention complete and to fully inform those skilled in the art of the scope of the invention. The present invention is defined only by the scope of the claims. Like reference numerals refer to like elements throughout the specification.

Unless otherwise defined, all terms (including technical and scientific terms) used in this specification will be used to have meanings that can be commonly understood by those skilled in the art to which the present invention pertains. Additionally, terms defined in commonly used dictionaries are not interpreted ideally or excessively unless clearly specifically defined. The terminology used herein is for describing embodiments and is not intended to limit the invention. As used herein, singular forms also include plural forms, unless specifically stated otherwise in the context.

The present invention provides a titanium material having a surface modified by plasma treatment, a dental titanium implant made of the titanium material, and a dental titanium implant having a surface modified by plasma treatment.

The surface-modified titanium material and dental titanium implant according to the present invention have improved cell proliferation ability, adhesion ability, and motility, and also have improved antibacterial effect. In addition, plastic deformation occurs on the surface, resulting in high chemical activity and reactivity, low C/O ratio on the surface, and thus high reactivity with cells.

The present invention also provides a method of modifying the surface of a titanium material, including the steps of: (a) filling a plasma generating device with a carrier gas: (b) generating plasma in the plasma generating device; and (c) irradiating the generated plasma to titanium.

According to another embodiment of the present invention, there is provided a method of manufacturing a surface-modified dental implant, including the steps of: (a) preparing an implant made of titanium material: (b) filling a plasma generating device with a carrier gas: (c) generating plasma in the plasma generating device; and (d) irradiating the generated plasma to the implant in step (a).

In the present invention, the titanium material or implant is preferably for medical use, more specifically for dental use.

The surface-modified titanium material and implant according to the present invention may have a surface C/O ratio of 0.1 to 0.2.

Additionally, the plasma of the present invention may be formed under atmospheric pressure conditions, and preferably, may include N₂ or N₂/Ar (consist of N₂ or N₂/Ar gas) as a carrier gas. Here, the N₂/Ar mixed gas preferably contains N₂ and Ar in a molar ratio of 1 to 5:5 to 50.

The material or the implant surface-modified according to the present invention may be treated with plasma for 0.1 second to 10 minutes.

The plasma of the present invention may be generated by supplying a voltage of 0.1 kV to 20 kV and a frequency of 10 to 30 kHz to the plasma generating device, and may be radiated under conditions of room temperature and atmospheric pressure. The plasma of the present invention is preferably irradiated to the titanium for 0.1 seconds to 10 minutes.

### Advantageous Effects

The surface-modified titanium material and titanium implant according to the present invention have improved cell proliferation ability, adhesion ability, and motility, and also have improved antibacterial effect.

In addition, the titanium material and titanium implant in the present invention are plasma treated and thus have reduced water contact angle and diodomethane contact angle, and undergo plastic deformation on the surface, resulting in high chemical activity and reactivity. In addition, the titanium material of the present invention has a low C/O ratio on the surface and thus is highly reactive with cells, making it suitable for use as a dental material.

The present invention provides the most effective type and generation method of plasma for surface modification of titanium materials.

According to the method of manufacturing a surface-modified titanium material and implant according to the present invention, there is an advantage in that the antibacterial properties and cell adhesion ability of the implant can be improved, thereby minimizing the inflammatory response caused by the use of the implant.

In addition, the implant according to the present invention has a hydrophilic moisturizing film formed, thereby improving the success rate of the implant procedure while shortening the procedure period.

The surface-modified titanium material or implant using the same according to the present invention may be used for purposes such as antibacterial (sterilization), cell proliferation, and cell adhesion.

### DESCRIPTION OF DRAWINGS

FIG. 1 is an image schematically illustrating a method of modifying a surface of a titanium material according to an embodiment of the present invention.

FIG. 2 shows the results of confirming the water contact angle after irradiating a titanium specimen with plasma composed of each of Ar, HeO₂, N₂, and N₂/Ar carrier gases according to an embodiment of the present invention.

FIG. 3 shows the results of confirming the diiodomethane contact angle after irradiating a titanium specimen with plasma composed of each of Ar, HeO₂, N₂, and N₂/Ar carrier gases according to an embodiment of the present invention.

FIG. 4 shows the results of separately confirming the surface free energy for a dispersion component and a polar component after irradiating a titanium specimen with plasma composed of each of Ar, HeO₂, N₂, and N₂/Ar carrier gases according to an embodiment of the present invention.

FIG. 5 shows the results of separately confirming three-dimensional surface texture parameters, in particular arithmetic mean height Sa; root mean square height, Sq; and maximum pit height Sv after irradiating a titanium specimen with plasma composed of each of Ar, HeO₂, N₂, and N₂/Ar carrier gases according to an embodiment of the present invention.

FIG. 6 shows a comparison of SEM images before and after irradiating a titanium specimen with plasma composed of each of Ar, HeO₂, N₂, and N₂/Ar carrier gases according to an embodiment of the present invention.

FIG. 7 shows the results of measuring the concentration of nitrogen (N) present on the surface of titanium after irradiating a titanium specimen with plasma composed of each of Ar, HeO₂, N₂, and N₂/Ar carrier gases according to an embodiment of the present invention.

FIG. 8 shows the results of measuring the concentration of oxygen (O) present on the surface of titanium after irradiating a titanium specimen with plasma composed of each of Ar, HeO₂, N₂, and N₂/Ar carrier gases according to an embodiment of the present invention.

FIG. 9 shows the results of the ratio of C/O present on the surface of titanium after irradiating a titanium specimen with plasma composed of each of Ar, HeO₂, N₂, and N₂/Ar carrier gases according to an embodiment of the present invention.

FIG. 10 shows the results of confirming the number of bacteria present on a titanium specimen after irradiating the titanium specimen with plasma composed of each of N₂, and N₂/Ar carrier gases according to an embodiment of the present invention.

FIG. 11 shows the results of confirming the effect of reducing the adhesion ability of Porphyromonas gingivalis (hereinafter referred to as P.gingivalis) on the surface of titanium after irradiating a titanium specimen with plasma composed of each of N₂, and N₂/Ar carrier gases according to an embodiment of the present invention.

FIG. 12 is a schematic diagram of a cell proliferation test experiment according to an embodiment of the present invention.

FIG. 13 shows the results of confirming the proliferation of osteoblasts over time after irradiating a titanium specimen with plasma composed of each of N₂, and N₂/Ar carrier gases according to an embodiment of the present invention.

FIG. 14 shows the results of confirming the motility of osteoblasts over time after irradiating a titanium specimen with plasma composed of N₂ carrier gas according to an embodiment of the present invention.

FIG. 15 shows the results of confirming the motility of osteoblasts over time after irradiating a titanium specimen with plasma composed of N₂/Ar carrier gas according to an embodiment of the present invention.

FIG. 16 shows the results of confirming the extracellular release of H₂O₂ from osteoblasts over time after irradiating a titanium specimen with plasma composed of each of N₂, and N₂/Ar carrier gases according to an embodiment of the present invention.

FIG. 17 shows the results of confirming the extracellular release of NO₂ from osteoblasts over time after irradiating the osteoblasts with plasma composed of each of N₂, and N₂/Ar carrier gases according to an embodiment of the present invention.

FIG. 18 shows the results of confirming the adhesion ability of osteoblasts over time after irradiating titanium with plasma composed of N₂/Ar carrier gas according to an embodiment of the present invention.

FIG. 19 shows the results of confirming the adhesion ability of osteoblasts over time after irradiating titanium with plasma composed of N₂ carrier gas according to an embodiment of the present invention.

### BEST MODES OF THE INVENTION

Hereinafter, the present invention will be described in detail by way of examples in order to aid understanding of the present invention. However, the following examples are only for illustrating the contents of the present invention, and the scope of the present invention is not limited to the following examples. The examples of the present invention are provided to explain the present invention more completely to those skilled in the art.

The present invention is to provide a titanium material having improved cell proliferation ability, adhesion ability and motility, and also having improved antibacterial effect. The titanium material of the present invention is plasma treated and thus has reduced water contact angle and diodomethane contact angle, and undergo plastic deformation on the surface, resulting in high chemical activity and reactivity. In addition, the titanium material of the present invention has a low C/0 ratio on the surface and thus is highly reactive with cells.

In the present invention, “plasma” can be generated by supplying a specific pressure, voltage, or frequency to a carrier gas. The “carrier gas” in the present invention may be at least one gas selected from the group consisting of nitrogen, helium, argon, and oxygen, but is not limited thereto. Preferably, it may be composed of a mixture of at least two gases among nitrogen, helium, argon, and oxygen, and most preferably, may be nitrogen (N₂) or a mixture of nitrogen and argon (N₂/Ar), but is not limited thereto. In the present invention, the mixture of nitrogen and argon may be a mixture of N₂ and Ar in a molar ratio of 1 to 5:5 to 50, preferably in a molar ratio of 1:9.

The plasma of the present invention can be generated by supplying a voltage of 0.1 kV to 20 KV and/or a frequency of 10 to 30 kHz to the carrier gas. Most preferably, it can be generated by applying a voltage of 5 kV and/or a frequency of 25 KHz.

In the present invention, “surface modification” refers to imparting physical, chemical, and biological properties that were not present in an original material to the surface of the material. In the present invention, by plasma treatment, the water contact angle and diodomethane contact angle on the surface of the titanium material are reduced, plastic deformation occurs on the surface, thereby increasing chemical activity and reactivity, lowering C/O ratio, and thus increasing reactivity with cells.

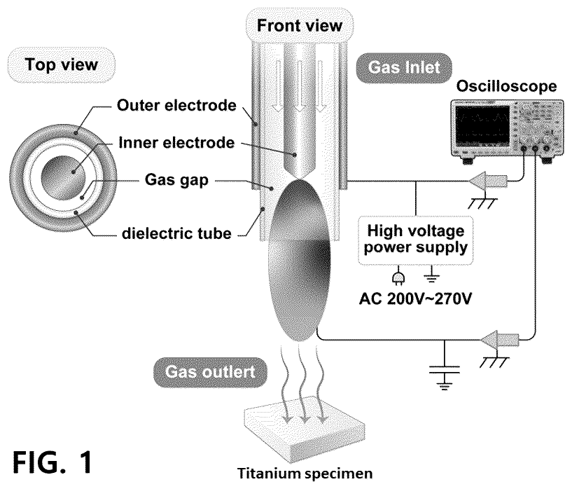

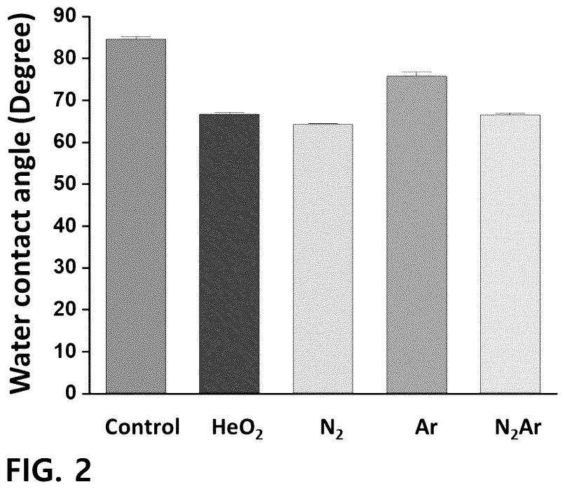

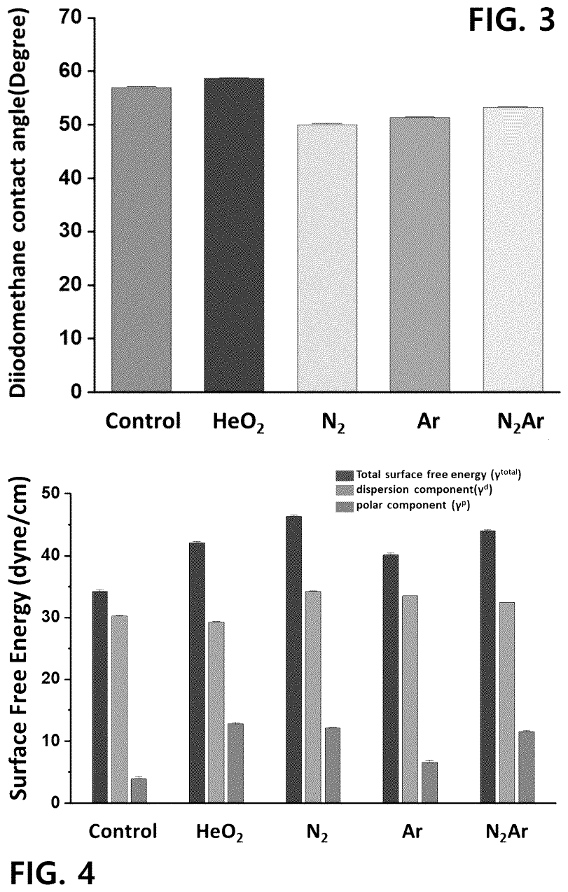

According to one embodiment of the present invention, the water contact angle and diiodomethane contact angle on the titanium surface are overall reduced by plasma treatment (see FIGS. 1 and 2). In particular, when treated with plasma, the surface free energy increases, and in particular, in the case of plasma composed of N₂ and N₂/Ar (as a carrier gas), the polar component (γ^{p}) increases (see FIG. 3), indicating that the surface-modified titanium material according to the present invention has improved cell adhesion ability.

In addition, according to an embodiment of the present invention, in the case of titanium whose surface was modified by plasma treatment, there is no significant difference in surface roughness, but in the case of plasma composed of N₂ (as a carrier gas), plastic deformation occurs, accelerating oxygen uptake on the surface of the modified material, and increasing chemical activity (chemical reactivity) and reactivity (kinetics of surface reactions).

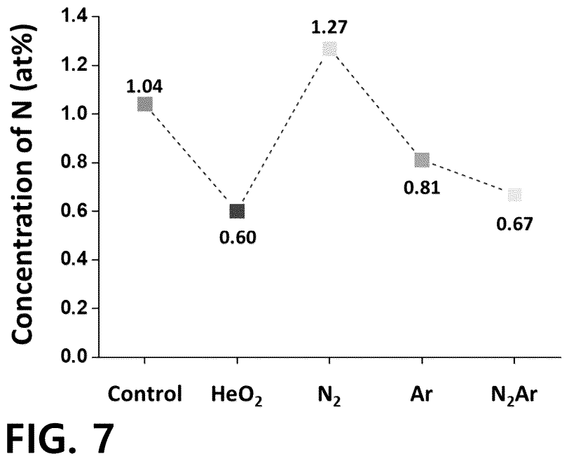

The composition of the surface of titanium whose surface has been modified according to the present invention may vary depending on plasma treatment. When treated with plasma using N₂ as a carrier gas according to an embodiment of the present invention, the carbon/oxygen (C/O) ratio on the titanium surface is 0.1 to 0.2 or 0.1 to 0.15, resulting in increased reactivity with cells (see FIGS. 7 to 9).

In the case of the titanium material whose surface is modified according to the present invention, the antibacterial effect increases and the adhesion ability of bacteria decreases, making it suitable for use as a dental material. The titanium material of the present invention has an antibacterial effect against bacteria existing in the oral cavity, and has an antibacterial (sterilizing) effect against at least one bacterium selected from the group consisting of Porphyromonas gingivalis, Tannerella forsythia, and Treponema denticola.

In addition, the titanium material manufactured according to the present invention has improved cell proliferation ability, cell motility, and cell adhesion ability in relation to cells (preferably, osteoblasts), making it suitable for use as a medical material. As described above, the titanium material of the present invention has improved cell proliferation ability, motility, and adhesion ability in reactions with osteoblasts, and thus, the effectiveness of bone formation and osseointegration is improved when used as a dental material.

In the present invention, “treatment” with plasma refers to irradiating titanium (or a specific material made of titanium) with plasma. In the present invention, the plasma may be radiated under conditions of room temperature and atmospheric pressure, and may be irradiated to the titanium at a distance of 10 mm for 0.1 seconds to 10 minutes, preferably 60 seconds.

The “titanium material” of the present invention may be used as a dental material, and may be used for implants, crowns, inlays, posts, and orthodontic brackets, and most preferably, as a dental implant material, but is not limited thereto.

A dental implant generally refers to a replacement that restores the original function of a tooth by implanting and adhering a fixture to an alveolar bone from which the natural tooth root has come out to replace the lost tooth root, and then fixing an artificial tooth on top of the fixture.

In the present invention, “dental titanium implant with a surface modified by plasma treatment” may be made of the titanium material with a modified surface, or may be manufactured by treating an implant made of titanium with plasma.

Hereinafter, the present invention will be described in more detail by way of examples. These examples are only for illustrating the present invention, and it is obvious to those skilled in the art that the scope of the present invention is not interpreted as limited by these examples.

### Statistical Analysis

Statistical significance of the data was assessed by one-way analysis of variance (ANOVA) with Tukey's honesty significant difference (HSD) post hoc test at α=0.05. All analyzes were performed using statistical software (IBM SPSS Statistics, v25.0, IBM Corp., Chicago, IL, USA).

### Example 1

### Sample Preparation and Plasma Surface Treatment

In the present invention, commercially pure (CP) grade 4 titanium was used. A total of 140 plate-shaped samples (10.0 mm×10.0 mm×1.0 mm) were prepared and polished with a uniform finish using 800 grit SiC paper. After ultrasonic washing for 20 minutes, plasma irradiation was performed at room temperature using a low-temperature atmospheric pressure DBD plasma generator (PR-ATO-001, ICD Co., Anseong, Gyeonggi-do, Korea). Plasma was applied perpendicular to the sample surface at a distance of 10 mm for 60 seconds. A schematic diagram of the device used in the experiment is shown in FIG. 1. All samples were randomly assigned to five groups (n=28), wherein four groups were treated with plasmas composed of four different gases (Ar, N₂, N₂/Ar mixture (10% nitrogen/90% argon), and He/O₂ mixture (15% helium/85% oxygen), and one group was used as a control without plasma treatment. The input voltage was fixed at 5 KV using a high-voltage transformer, and the operating frequency was set to 25 kHz using a digital oscilloscope (MSO4032, Tektronix, Beaverton, OR, USA). The mass flow controller maintained a constant gas flow rate of 10 standard liters per minute (slm).

### Example 2

Analysis of Properties of Titanium Surface after Plasma Treatment 2-1. Confirmation of Changes in Surface Contact Angle and Surface Free Energy According to Plasma Treatment

The surface wettability of the samples was measured using a contact angle meter (Phoenix 300 Touch, S.E.O., Suwon, Gyeonggi-do, Korea). The contact angle was measured by the sessile drop technique at room temperature and 60% relative humidity using distilled water (n=10) and nonpolar diiodomethane (n=10), and all measurements were performed at the center of the samples.

The surface free energy was calculated by measuring the contact angle of two liquids (distilled water and nonpolar diodomethane) according to the Owens-Wendt equation. The total surface free energy (γtotal) including the dispersion component (γ^{d}) and the polar component (γ^{p}) was calculated.

FIG. 2 shows the water contact angle when treated with plasma according to this embodiment, and FIG. 3 shows the diiodomethane contact angle when treated with plasma according to this embodiment. Referring to FIGS. 2 and 3, it can be seen that there was a difference in contact angles according to the plasma, and in the case of Ar, HeO₂, N₂, N₂/Ar plasma treatment, the water contact angle decreased, while in the case of Ar, N₂, N₂/Ar plasma treatment, the diiodomethane contact angle decreased.

FIG. 4 shows the total surface free energy (γ^{total}) and the respective values of γ^{d} and γ^{p} of titanium samples treated with plasma composed of different types of gas. Referring to FIG. 4, the total surface energy was significantly increased after plasma treatment in all samples, and in particular, the greatest increase in surface free energy was observed in N₂ and N₂Ar. It can be seen that among the surface free energies, the polar component (γ^{p}), which is highly related to cell adhesion, was increased in N₂, N₂Ar, and HeO₂ plasmas. That is, it can be seen that according to this example, when the titanium sample is treated with plasma, the water contact angle and the diodomethane contact angle decrease, and the surface free energy is high in N₂ and N₂Ar, and in particular, when treated with N₂ plasma, the value of γ^{p} is significantly increased, thereby improving the cell adhesion ability of the titanium samples. This indicates that the surface of the titanium sample was modified by plasma treatment.

### 2-2. Surface Shape Change

Three-dimensional (3-D) surface properties were analyzed using a confocal laser scanning microscope (CLSM; LEXT OLS3000, Olympus, Tokyo, Japan) at 50× magnification in an area of 256×192 μm². Surface texture parameters, in particular arithmetic mean height Sa; root mean square height, Sq; and maximum pit height Sv were calculated according to ISO 25,178. Surface analysis was performed independently at two locations in the center, and a total of 10 measurements were made for each sample treated with plasma composed of different types of gases.

The surface microstructure of the samples was evaluated using a scanning electron microscope (SEM: JSM-7800F Prime, JEOL, Tokyo, Japan) at an acceleration voltage of 5.0 kV and a working distance (WD) of 6.0 mm at 3000, 10,000, and 30,000×magnification (n=1).

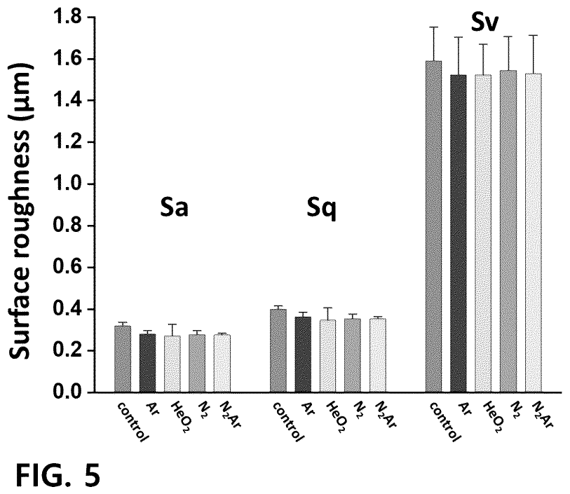

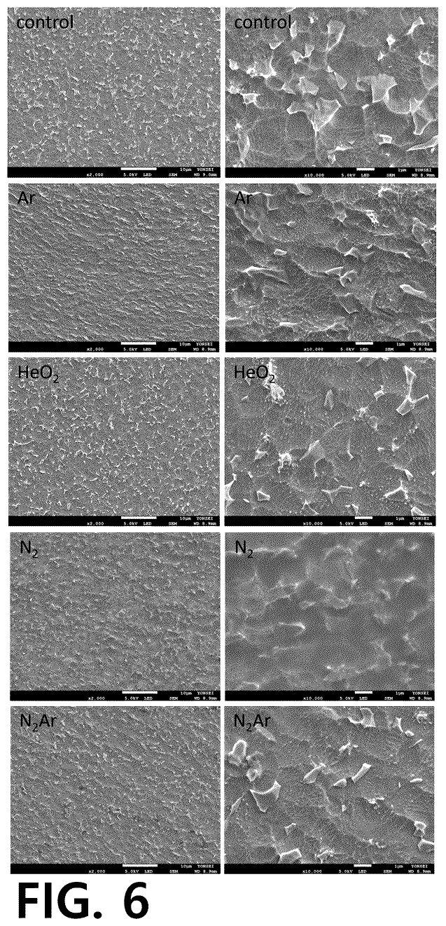

Enlarged confocal images and SEM images of samples treated with plasma composed of different types of gases are shown in FIG. 6. The surface texture parameters Sa, Sq and Sv measured in CLSM are shown in FIG. 5.

Referring to FIG. 5, it can be seen that as a result of the surface roughness analysis, there is no statistically significant difference between the roughness of the titanium surface after plasma treatment and the roughness before treatment. As a result of confirming the SEM images after each plasma treatment in FIG. 6, the most plastic deformation was observed on the surface of the N₂ plasma-treated titanium. This suggests that the plastic deformation can induce atomic diffusion into gaps and accelerate oxygen uptake on the titanium surface, thereby increasing chemical activity (chemical reactivity) and reactivity (kinetics of surface reactions).

### 2-3. Confirmation of Chemical Changes

The elemental composition of titanium samples treated with plasma composed of different types of gases was analyzed using a monochromatic Al Kα X-ray source (1486.6 eV) at 12 kV and 3 mA through X-ray photoelectron spectroscopy (XPS) (K-alpha, Thermo Fisher Scientific Inc., Waltham, MA, USA) (n=1). Additionally, data were collected and core-level spectra were analyzed using software (Thermo Avantage v5.980, Thermo Fisher Scientific Inc., Waltham, MA, USA). All XPS spectra were calibrated to a C Is peak at 284.6 eV.

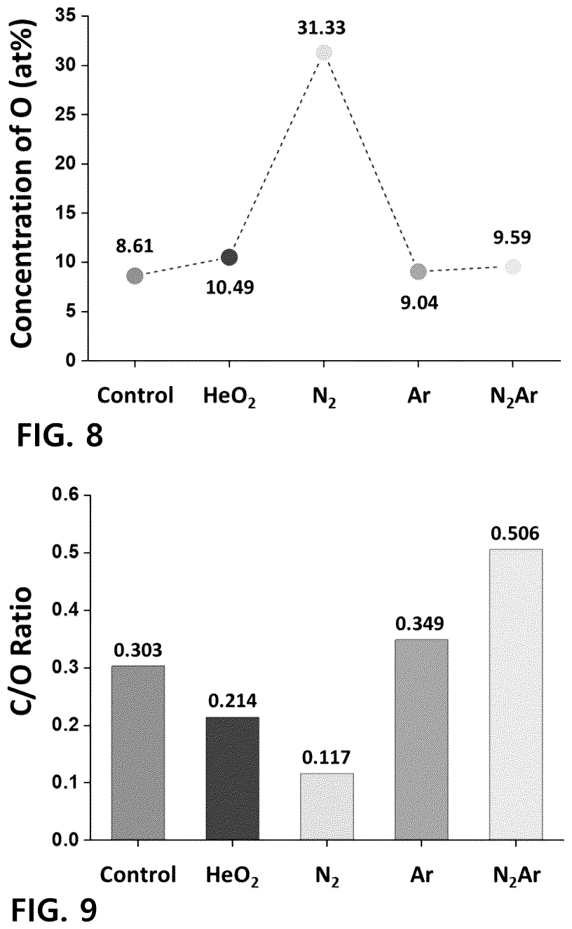

FIGS. 7 and 8 show the atomic percentages (at %) of nitrogen and oxygen, respectively, as determined by XPS, and FIG. 9 shows the carbon/oxygen ratios in all samples. In all samples, the nitrogen content on the titanium surface after plasma treatment was increased compared to the control when treated with plasma composed of N₂, and the nitrogen content was decreased when treated with plasma composed of Ar, HeO₂, and N₂/Ar. It can be seen that the oxygen content also increased significantly compared to the control when treated with plasma composed of N₂.

In conclusion, referring to FIG. 9, it can be seen that the value of the C/O ratio is lowered in the case of the titanium sample whose surface was modified by plasma treatment according to the present invention, suggesting that the titanium surface treated with plasma composed of N₂ gas has high reactivity with cells.

### Example 3

### Confirmation of P.Gingivalis Sterilization Effect on Titanium Surface by Plasma Treatment

In this example, as a result of analyzing the characteristics of the titanium surface treated with each of four types of plasma (Ar, HeO₂, N₂, N₂/Ar) for 1 minute, two types of plasma (N₂, N₂Ar) with the highest surface energy were selected, and the sterilizing power of each plasma against P.gingivalis bacteria was measured.

After P.gingivalis was smeared on the titanium surface, each plasma was irradiated and the number of bacteria over time was measured.

The experimental process is as follows. First, the bacteria stored in a deep freezer were taken out and thawed at room temperature. The stored bacteria were diluted in 1 mL TSB medium (tryptic soy broth) so as to become 2*105 bacteria. Thereafter, they were washed twice with DW/PBS (all of glycerol, a storage solvent, was removed through the washing process) (1 ml of DW->vortex->removal of DW was repeated twice). After washing, 1 ml of TSB medium (tryptic soy broth) was added and mixed well so that the bacteria were homogeneous. Afterward, 100 μl was picked (104) and smeared on the medium using a spreader. After smearing, it was treated with plasma according to each plasma treatment time (10 minutes, 20 minutes, 30 minutes) and then incubated at a temperature of 37 degrees.

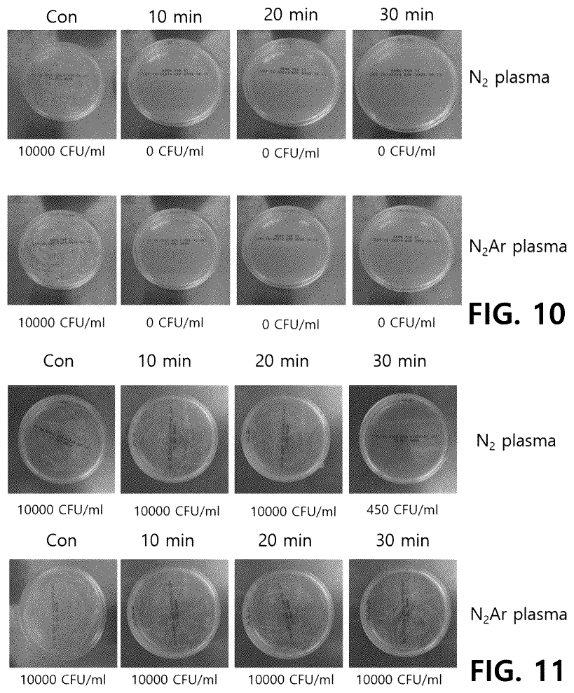

Before counting the number of bacteria in this example, 2 to 3 plates were smeared for each sample and then each plate was counted. The control sample was set to grow 10,000 colonies on the plate through several experiments, and then the experiment was performed in the same amount. Counting (counting 100 or more): For each plate, the bacteria within 1 cm² were counted according to the dense plate measurement method and then multiplied by 65 to calculate the number of bacteria on the plate, which was shown in FIG. 10, and the unit was cfu/ml.

Referring to FIG. 10, in the case of both N₂ and N₂Ar plasma, there were no bacteria (zero) remaining on the titanium specimen when treated with plasma for 10 minutes. Therefore, it can be confirmed that both types of plasma have a sterilization effect of about 100% when treated for 10 minutes.

### Example 4

### Confirmation of the Effect of Plasma Treatment on Reducing P.Gingivalis Adhesion to the Titanium Surface

In this example, the effect of reducing the adhesion of P.gingivalis to the titanium surface treated with two types of plasma (N₂, N₂/Ar) was measured.

First, a certain amount of P.gingivalis was applied to titanium specimens that had been treated with N₂ and N₂/Ar plasma for 10, 20, and 30 minutes, respectively, and reacted for about 30 minutes. Afterward, the bacteria in the reacted specimens were washed, placed in a culture medium, and smeared on a solid medium. The smeared medium was cultured for one day to measure the bacterial CFU (number of viable microorganisms per 1 mL (Colony Forming Unit, CFU)). The results are shown in FIG. 11.

Referring to FIG. 11, no decrease in P.gingivalis adhesion was found in the titanium specimens treated with N₂/Ar plasma for 10, 20, and 30 minutes and N₂ plasma for 10 and 20 minutes, but 95.5% reduction in P.gingivalis adhesion was found in the titanium specimens treated with N₂ plasma for 30 minutes (4.5% remained). That is, it can be seen that when treated with the plasma made of N₂ gas, there is an effect of weakening the adhesion of bacteria on the surface of the material.

### Example 5

### Confirmation of Osteoblast Proliferation (Cell Proliferation) on Titanium Surface by Plasma Treatment

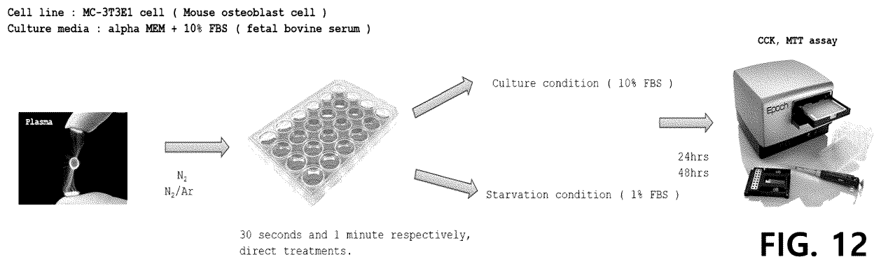

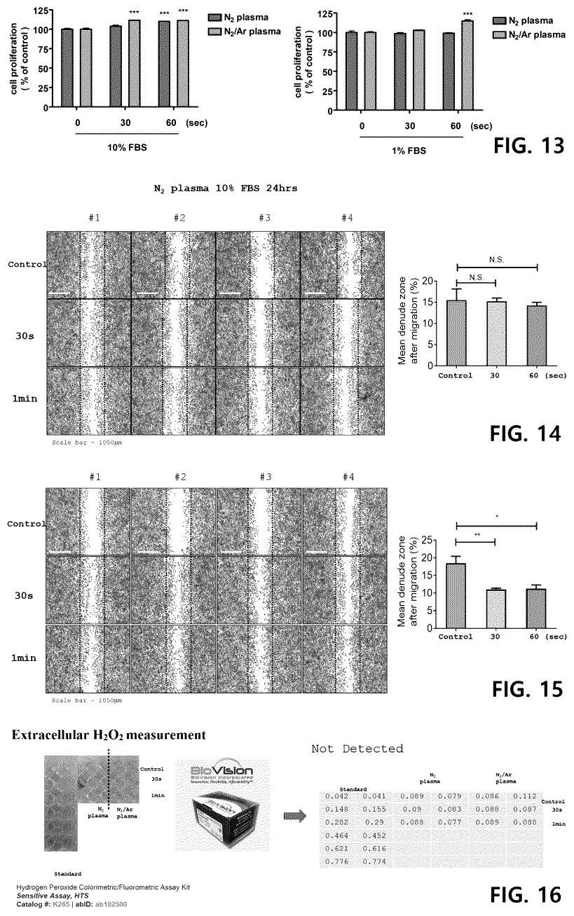

When each titanium sample was treated with N₂ or N₂/Ar plasma for 30 seconds under 10% FBS conditions, approximately 0.4% of osteoblasts were proliferated in the case of plasma composed of N₂ compared to the control, and 11% of osteoblasts were proliferated in the case of N₂/Ar plasma compared to the control. In addition, when treated with the plasma for 60 seconds, it was confirmed that 10% (N₂) and 11% (N₂/Ar) of osteoblasts were proliferated, respectively (see FIGS. 12 and 13).

It could be confirmed that under the 1% FBS condition, there was no statistical significance in the N₂ condition compared to the control, and 0.3% and 15% of osteoblasts were proliferated under the N₂/Ar condition, respectively.

That is, it can be seen that the N₂ or N₂/Ar plasma can contribute to cell proliferation activation of osteoblasts.

### Example 6

### Confirmation of Osteoblast Motility on Titanium Surface by Plasma Treatment

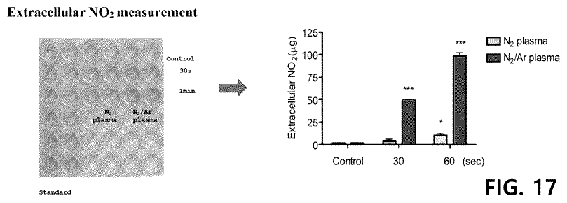

In this example, the motility (migration effect) of osteoblasts on the titanium surface treated with N₂ or N₂/Ar plasma was confirmed. When treated with N₂ plasma for 30 or 60 seconds according to this example, no effect was observed (see FIG. 14), but when treated with N₂Ar plasma for 30 seconds, the migration of osteoblasts was confirmed (FIG. 15). In this case, however, it can be confirmed that extracellular H₂O₂ did not appear (see FIG. 16), but when comparing extracellular NO₂ over time, the NO₂ was released in both N₂ and N₂Ar plasma, and in particular, it was expressed significantly higher in N₂Ar than in N₂ (see FIG. 17).

That is, it can be seen that according to this example, the cell growth and motility of osteoblasts are improved on the titanium surface treated with N₂ or N₂/Ar plasma.

### Example 7

### Confirmation of Osteoblast Adhesion Ability on Titanium Surface by Plasma Treatment

In this example, MC-3T3E1 cell (mouse osteoblast cell) was used as a cell line in order to confirm the osteoblast adhesion ability on the titanium surface by plasma treatment. Alpha MEM+10% fetal bovine serum (FBS) was used as a culture medium, and each sample was pretreated with N₂ or N₂Ar plasma for 10 and 20 minutes, respectively, and then 2X10⁴ cells were seeded. Afterward, SYTOX staining (green staining (nucleus stain, 1:30,000)) was performed for 15 minutes, and the cell adhesion ability was confirmed by dividing the field of the titanium sample.

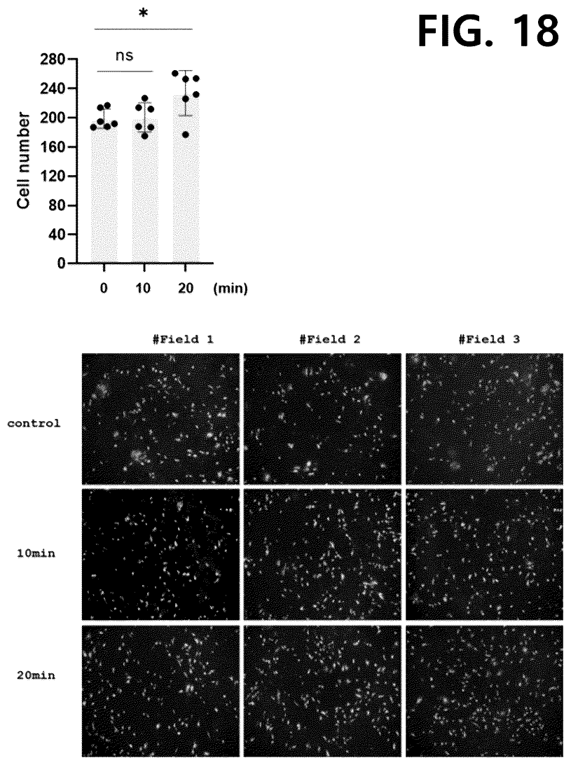

Referring to FIG. 18, there was no change in titanium treated with N₂/Ar plasma for 10 minutes, but the adhesion of ostroblast was increased in titanium treated for 20 minutes.

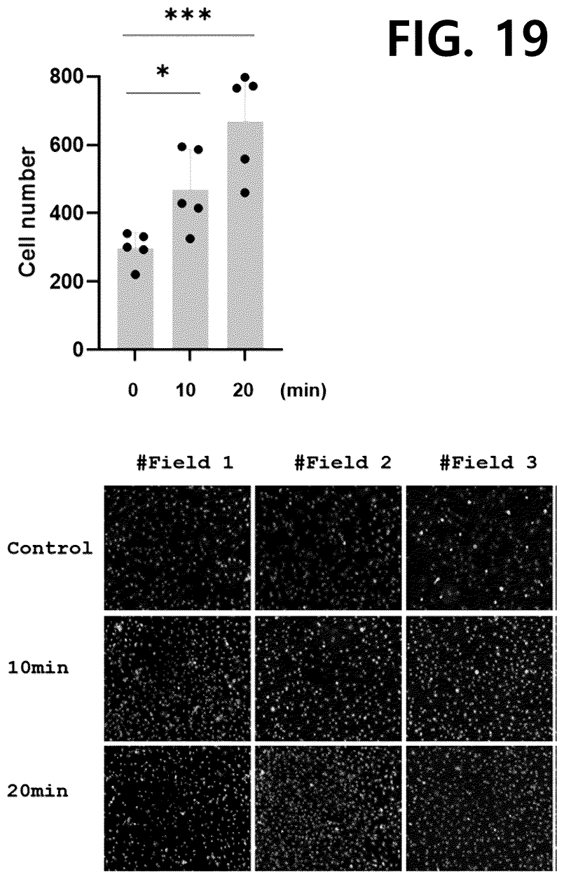

Referring to FIG. 19, when treated with N₂ plasma, the osteoblast adhesion increased after 10 minutes of treatment, and the number of increased cells was greater than in the case of N₂/Ar plasma.

In other words, it can be seen that the adhesion ability of osteoblasts appears in both N₂ plasma and N₂/Ar plasma, but is higher in N₂ plasma.

It is to be understood that the description of the present invention described above is for illustrative purposes only, and can be easily modified into other specific embodiments by those of ordinary skill in the art to which the present invention pertains without changing the technical spirit or essential features of the present invention. Therefore, it should be understood that the examples described above are illustrative in all respects and not restrictive.

The invention claimed is:

1. A titanium material with a surface modified by plasma treatment.

2. The titanium material with a surface modified according to claim 1, wherein the material is for dental use.

3. The titanium material with a surface modified according to claim 1, wherein the material has a surface C/O ratio of 0.1 to 0.2.

4. The titanium material with a surface modified according to claim 1, wherein the plasma is a plasma formed under atmospheric pressure conditions.

5. The titanium material with a surface modified according to claim 1, wherein the plasma contains N₂ or N₂/Ar as a carrier gas.

6. The titanium material with a surface modified according to claim 5, wherein the N₂/Ar mixed gas contains N₂ and Ar in a molar ratio of 1 to 5:5 to 50.

7. The titanium material with a surface modified according to claim 1, wherein the material is treated with plasma for 0.1 second to 10 minutes.

8. A dental titanium implant with a surface modified by plasma treatment.

9. The dental titanium implant according to claim 8, wherein the implant has a surface C/O ratio of 0.1 to 0.2.

10. The dental titanium implant according to claim 8, wherein the plasma is a plasma formed under atmospheric pressure conditions.

11. The dental titanium implant according to claim 8, wherein the plasma contains N₂ or N₂/Ar as a carrier gas.

12. A method of modifying the surface of a titanium material, including the steps of:
(a) filling a plasma generating device with a carrier gas;
(b) generating plasma in the plasma generating device; and
(c) irradiating the generated plasma to titanium.

13. The method of modifying the surface of a titanium material according to claim 12, wherein the surface-modified titanium material has a surface C/O ratio of 0.1 to 0.2.

14. The method of modifying the surface of a titanium material according to claim 12, wherein the material is for dental use.

15. The method of modifying the surface of a titanium material according to claim 12, wherein the carrier gas is N₂ or N₂/Ar.

16. The method of modifying the surface of a titanium material according to claim 12, wherein the plasma in step (b) is generated by supplying a voltage of 0.1 kV to 20 kV and a frequency of 10 to 30 kHz to the plasma generating device.

17. The method of modifying the surface of a titanium material according to claim 12, wherein the plasma generated in step (b) is radiated under conditions of room temperature and atmospheric pressure.

18. The method of modifying the surface of a titanium material according to claim 12, wherein the plasma in step (c) is irradiated to the titanium for 0.1 seconds to 10 minutes.

19. A method of manufacturing a surface-modified dental implant, including the steps of:
(a) preparing an implant made of titanium material;
(b) filling a plasma generating device with a carrier gas;
(c) generating plasma in the plasma generating device; and
(d) irradiating the generated plasma to the implant in step (a).

20. The method of manufacturing a surface-modified dental implant according to claim 19, wherein the surface-modified dental implant has a surface C/O ratio of 0.1 to 0.2.

21. The method of manufacturing a surface-modified dental implant according to claim 19, wherein the carrier gas is N₂ or N₂/Ar.

22. The method of manufacturing a surface-modified dental implant according to claim 19, wherein the plasma in step (c) is generated by supplying a voltage of 0.1 kV to 20 kV and a frequency of 10 to 30 kHz to the plasma generating device.

23. The method of manufacturing a surface-modified dental implant according to claim 19, wherein the plasma generated in step (c) is radiated under conditions of room temperature and atmospheric pressure.

24. The method of manufacturing a surface-modified dental implant according to claim 19, wherein the plasma in step (d) is irradiated to the implant for 0.1 seconds to 10 minutes.
